(19) United States
(12) Patent Application Publication
(10) Pub. No.: US 2012/0184859 A1
(43) Pub. Date: Jul. 19, 2012

(54) SYSTEMS AND METHODS FOR CORROBORATING IMPEDANCE-BASED LEFT ATRIAL PRESSURE (LAP) ESTIMATES FOR USE BY AN IMPLANTABLE MEDICAL DEVICE

(75) Inventors: Riddhi Shah, Mountain View, CA (US); Fujian Qu, Sunnyvale, CA (US); Yelena Nabutovsky, Sunnyvale, CA (US); Dan E. Gutfinger, Agoura Hills, CA (US); Ryan Rooke, Redondo Beach, CA (US); Alex Soriano, Ventura, CA (US)

(73) Assignee: PACESETTER, INC., Sylmar, CA (US)

(21) Appl. No.: 13/007,364

(22) Filed: Jan. 14, 2011

(51) Int. Cl. A61B 5/0215; A61B 5/021

(52) U.S. Cl. 600/486; 600/485

(57) ABSTRACT

Various techniques are provided for assessing the reliability of left atrial pressure (LAP) estimates made by an implantable medical device based on impedance values or related electrical values. In one example, various cardioelectric and cardiomechanical parameters are used to corroborate LAP estimation in circumstances where the LAP estimates deviate from an acceptable, satisfactory or otherwise healthy range. The cardioelectric parameters include, e.g.: ST elevation; heart rate (HR); heart rate variability (HRV); T-wave alternans (TWA); QRS waveform parameters; P-wave duration; evoked response (ER) parameters; and intrinsic PV/AV/VV conduction delays. The cardiomechanical parameters include, e.g.: heart rate turbulence (HRT); cardiogenic impedance signals; heart sounds; and non-LAP blood pressure measurements, such as aortic pressure measurements. The device compares the cardioelectric and cardiomechanical parameters against corresponding baseline values to determine whether variations in the parameters corroborate the LAP estimates. If not, the LAP estimates are selectively cancelled or suspended, or the overall procedure is re-calibrated.

### RELATED APPLICATIONS

This application is related to U.S. patent application Ser. No. ______, filed ______, entitled “Systems and Methods for Exploiting Near-Field Impedance and Admittance for Use with Implantable Medical Devices”.

### FIELD OF THE INVENTION

The invention relates to implantable medical devices such as pacemakers, implantable cardioverter defibrillators (ICDs) and cardiac resynchronization therapy (CRT) devices and in particular to impedance-based techniques for use by such devices to estimate left atrial pressure (LAP) for use in detecting heart failure, pulmonary edema or related conditions.

### BACKGROUND OF THE INVENTION

Heart failure is a debilitating disease in which abnormal function of the heart leads to inadequate blood flow to fulfill the needs of the tissues and organs of the body. Typically, the heart loses propulsive power because the cardiac muscle loses capacity to stretch and contract. Often, the ventricles do not adequately fill with blood between heartbeats and the valves regulating blood flow become leaky, allowing regurgitation or back-flow of blood. The impairment of arterial circulation deprives vital organs of oxygen and nutrients. Fatigue, weakness and the inability to carry out daily tasks may result. Not all heart failure patients suffer debilitating symptoms immediately. Some may live actively for years. Yet, with few exceptions, the disease is relentlessly progressive. As heart failure progresses, it tends to become increasingly difficult to manage. Even the compensatory responses it triggers in the body may themselves eventually complicate the clinical prognosis. For example, when the heart attempts to compensate for reduced cardiac output, it adds cardiac muscle causing the ventricles to grow in volume in an attempt to pump more blood with each heartbeat, i.e. to increase the stroke volume. This places a still higher demand on the heart's oxygen supply. If the oxygen supply falls short of the growing demand, as it often does, further injury to the heart may result, typically in the form of myocardial ischemia or myocardial infarction. The additional muscle mass may also stiffen the heart walls to hamper rather than assist in providing cardiac output. Often, electrical and mechanical dyssynchronies develop within the heart such that the various chambers of the heart no longer beat in a synchronized manner, degrading overall cardiac function.

A particularly severe form of heart failure is congestive heart failure (CHF) wherein the weak pumping of the heart or compromised filling leads to build-up of fluids (i.e. congestives) in the lungs and other organs and tissues. The accumulation of fluids in the lungs to heart failure is referred to herein as cardiogenic pulmonary edema (PE). Briefly, the poor cardiac function resulting from heart failure can cause blood to back up in the lungs, thereby increasing blood pressure in the lungs, particularly pulmonary venous pressure. The increased pressure pushes fluid—but not blood cells—out of the blood vessels and into lung tissue and air sacs (i.e. the alveoli). This can cause severe respiratory problems and, left untreated, can be fatal. Note that noncardiogenic forms of PE can arise due to factors besides heart failure, such as infection. More specifically, noncardiogenic PE can be caused by changes in permeability of the pulmonary capillary membrane as a result of either a direct or an indirect pathologic insult.

Many patients susceptible to CHF and cardiogenic PE, particularly the elderly, have pacemakers, ICDs, CRT devices or other implantable medical devices implanted therein, or are candidates for such devices. Accordingly, it is desirable to provide techniques for detecting and tracking CHF and cardiogenic PE using such devices. One particularly effective parameter for detecting and tracking CHF is cardiac pressure, particularly LAP, i.e. the blood pressure within the left atrium of the patient. Reliable detection of LAP would not only permit the implanted device to track CHF/PE for diagnostic purposes but to also control therapies applied to address CHF/PE such as CRT. In this regard, CRT seeks to normalize asynchronous cardiac electrical activation and the resultant asynchronous contractions by delivering synchronized pacing stimulus to the ventricles using pacemakers, ICDs or CRT devices equipped with biventricular pacing capability. The pacing stimulus is typically synchronized so as to help to improve overall cardiac function. This may have the additional beneficial effect of reducing the susceptibility to life-threatening tachyarrhythmias. CRT and related therapies are discussed in, for example, U.S. Pat. No. 6,643,546 to Mathis et al., entitled “Multi-Electrode Apparatus And Method For Treatment Of Congestive Heart Failure”; U.S. Pat. No. 6,628,988 to Kramer et al., entitled “Apparatus And Method For Reversal Of Myocardial Remodeling With Electrical Stimulation”; and U.S. Pat. No. 6,512,952 to Stahmann et al., entitled “Method And Apparatus For Maintaining Synchronized Pacing”. Reliable estimates of LAP would also allow the dosing of heart failure medications (such as diuretics) to be properly titrated so as to minimize the number of episodes of acute heart failure decompensation. That is, accurate LAP monitoring can provide for the early identification of incipient HF decompensation and guide the adjustment of vasodilator and diuretic dosing.

However, LAP is a difficult parameter to detect since it is not clinically appealing to place a blood pressure sensor directly in the left atrium due to the chronic risk of thromboembolic events, as well as risks associated with the trans-septal implant procedure itself. Accordingly, various techniques have been developed for estimating LAP based on other parameters that can be more safely sensed by a pacemaker or ICD. In this regard, some particularly promising techniques have been developed that use electrical impedance signals (or related electrical signals such as admittance) to estimate LAP. For example, impedance signals can be sensed along a sensing vector passing through the left atrium, such as between an electrode mounted on a left ventricular (LV) lead and another electrode mounted on a right atrial (RA) lead. The sensed impedance is affected by the blood volume inside the left atrium, which is in turn reflected by the pressure in the left atrium (at least in accordance with a far-field interpretation of impedance signals.) Accordingly, there is a correlation between the sensed impedance and LAP, which can be exploited to estimate LAP and thereby also detect and/or track CHF and warn of cardiogenic PE.

For LAP estimation techniques based on impedance or related electrical parameters see: U.S. patent application Ser. No. 11/559,235, filed Nov. 13, 2006, of Gutfinger et al., entitled “System and Method for Estimating Cardiac Pressure Using Parameters Derived from Impedance Signals Detected by an Implantable Medical Device” (Attorney Docket No. A06P3024US06), now U.S. Pat. No. 7,794,404, which is incorporated by reference herein, as well as U.S. patent application Ser. Nos. 11/558,101 (Attorney Docket No. A06P3024US1); 11/557,851 (Attorney Docket No. A06P3024US2); 11/557,870, filed Nov. 8, 2006 (Attorney Docket No. A06P3024US3); 11/557,882, filed Nov. 8, 2006 (Attorney Docket No. A06P3024US4); and 11/558,088, filed Nov. 9, 2006 (Attorney Docket No. A06P3024US5), each entitled “Systems and Methods to Monitor and Treat Heart Failure Conditions.” See, also, U.S. patent application Ser. No. 11/558,194, filed Nov. 9, 2006, by Panescu et al., entitled “Closed-Loop Adaptive Adjustment of Pacing Therapy based on Cardiogenic Impedance Signals Detected by an Implantable Medical Device” (Attorney Docket No. A06P3031). Still further, see U.S. patent application Ser. No. 12/109,304, filed Apr. 25, 2008, of Gutfinger et al., entitled “System and Method for Calibrating Cardiac Pressure Measurements Derived from Signals Detected by an Implantable Medical Device” (Attorney Docket No. A08P1013), now U.S. Published Application No. 20080262361A1.

At least some of these documents describe cardiac pressure estimation techniques wherein a linear correlation between LAP and impedance (Z)—or related electrical signals such as admittance (Y) or conductance (G)—is exploited by the implanted device to estimate LAP. Briefly, the electrical signals are measured along a sensing vector passing through the heart of the patient in response to impedance-detection pulses generated by the device. Suitable conversion factors are determined via linear regression (or other suitable techniques including nonlinear regression) to relate the particular measured electrical signal parameter to LAP, so that measurements can then be used to estimate LAP. In one particular example, the conversion factors are “slope” and “baseline” values representative of the linear correlation between LAP and electrical parameter values measured in response to the impedance-detection pulses. Slope may also be referred to as “gain.” Baseline may also be referred to as “offset” or bLAP (i.e. baseline LAP.) Thereafter, LAP is estimated based using:

zLAP=Parameter*Slope+Baseline

wherein “Parameter” is the electrical parameter (converted to the same unit as LAP) measured in response to the impedance detection pulses, and zLAP represents the estimated LAP. Note that for the sake of generality, the term zLAP is used herein to refer to estimated LAP values whether based on actual impedance signals, or any of the related electrical signals such as admittance or conductance.

Although the foregoing techniques are helpful, there remains room for further improvement. One concern with using zLAP estimates is that the detected signals (impedance, admittance, etc.) may vary in response to non-LAP related factors, such as the presence of noncardiogenic PE, which might introduce significant errors into the LAP estimate. For example, during the early phase of noncardiogenic PE, actual LAP might remain at a regular or nominal level, whereas measured impedance signals can show a large decrease, particularly if the detection vector crosses a significant portion of pulmonary tissue. As a result, zLAP values estimated from impedance can be incorrect, possibly leading to inappropriate pacing therapy or the incorrect titration of drug dosages.

Accordingly, it is desirable to provide techniques for corroborating zLAP estimates in circumstances where the estimates might be inaccurate. It is to this end that the invention is primarily directed.

### SUMMARY OF THE INVENTION

In accordance with an exemplary embodiment of the invention, a method is provided for use with an implantable medical device—such as a pacemaker, ICD or CRT device—for corroborating the reliability of cardiac pressure estimates made by the implantable device based on impedance or related electrical values such as admittance or conductance. Briefly, LAP or other forms of cardiac pressure are estimated within the patient using impedance pulses or other electrical field signals applied to tissues in the thoracic domain of the patient. Again, for the sake of generality, the term zLAP is used herein to refer to LAP values estimated based on impedance or related electrical signals. One or more additional cardiac parameters are measured within the patient, such as cardioelectric parameters measured within an intracardiac electrogram (IEGM) or cardiomechanical parameters measured using physiological sensors. The reliability of the estimate of cardiac pressure is assessed using the measured cardiac parameters and then, based on the assessment of reliability, further estimates of cardiac pressure are selectively controlled to, for example, disable the use of the estimates for controlling therapy in circumstances where the estimates are deemed unreliable. That is, the additional cardiac parameters are used to corroborate zLAP estimates and, in the absence of that corroboration, zLAP can be cancelled, suspended or re-measured. The reliability assessment/corroboration techniques described herein are advantageously exploited in assessing the reliability of zLAP estimates in view of confounding influences such as noncardiogenic PE but can also be applied to assessing the reliability of other cardiac pressure estimates affected by other confounding factors.

In an illustrative implementation, the zLAP corroboration procedure is only performed if zLAP estimates are found to deviate from a predetermined range of acceptable, healthy or “nominal” values, such as a range extending up to 25 mmHg. In this regard, cardiogenic changes within the patient that can cause high LAP—such as changes due to CHF, cardiogenic PE, mitral regurgitation (MR), ischemia, etc.—are expected to also affect various cardioelectric and cardiomechanical parameters and hence these parameters can be used to corroborate or confirm the high zLAP estimate. That is, concordant changes among the various cardioelectric and cardiomechanical parameters can be used to corroborate zLAP. In one particular example, the corroboration assessment is performed whenever zLAP deviates from a predetermined acceptable range. In another example, the corroboration assessment is performed only if zLAP persistently falls outside the range. In either case, if zLAP is not corroborated by the cardioelectric and cardiomechanical parameters, further zLAP estimates are suspended or cancelled, at least until subsequent measurements indicate that the estimates are once again reliable. If zLAP suspensions/cancellations are found to be persistent indicating a chronic estimation problem, the device preferably deactivates zLAP pending clinician review or recalibrates itself, if so equipped.

In the illustrative implementation, the cardioelectric parameters used to corroborate zLAP when it exceeds the acceptable range include, e.g.: ST elevation; heart rate (HR); heart rate variability (HRV); T-wave alternans (TWA); QRS waveform parameters; P-wave duration; evoked response (ER) parameters; and intrinsic PV/AV/VV conduction delays. The cardiomechanical parameters include, e.g.: heart rate turbulence (HRT); an acceleration index; non-left atrial (LA) cardiogenic impedance signals; heart sounds and non-LAP blood pressure measurements, such as aortic pressure measurements or LV or RV pressure measurements. The device compares these various parameters against corresponding baseline values to determine whether the parameters corroborate zLAP estimates. In one particular example, the device generates an index representative of a cumulative difference between the cardioelectric and cardiomechanical measurements and their baseline values and then compares the index against a corroboration threshold. If the index exceeds the threshold (indicating significant changes in cardioelectric and cardiomechanical parameters consistent with abnormally high zLAP), the zLAP estimate is thereby corroborated and zLAP can be used by the device to control therapy and titrate medications. Conversely, if the index remains below the corroboration threshold, the zLAP estimates are deemed unreliable in view of possible confounding influences (such as noncardiogenic PE) and the use of zLAP to control therapy or titrate medications is suspended or cancelled. In one example, the index is calculated using:

Index=Σwᵢ*ΔCᵢ

where, C₁ is an i^{th} cardiac parameter measurement, ΔCᵢ is a difference between the i^{th} cardiac measurement and its corresponding baseline value, and wᵢ is a weight of Cᵢ. That is, different weights can be applied to the various cardioelectric and cardiomechanical parameters. Whenever zLAP is cancelled or suspended, suitable warning signals can be generated, including signals warning of a possible noncardiogenic PE in the patient. In some examples, if zLAP is deactivated due to persistent unreliability, a lead integrity test is triggered to detect possible lead failure.

Thus, various techniques are provided for corroborating or confirming zLAP estimates or other cardiac pressure estimates. Various method, system and apparatus examples of these techniques are described below.

### BRIEF DESCRIPTION OF THE DRAWINGS

Further features and advantages of the invention may be more readily understood by reference to the following description taken in conjunction with the accompanying drawings, in which:

FIG. 1 is a stylized representation of an exemplary implantable medical system equipped with a zLAP confirmation system;

FIG. 2 provides an overview of techniques for corroborating zLAP that may be performed by the system of FIG. 1;

FIG. 3 summarizes a first illustrative technique performed in accordance with the general method of FIG. 4, wherein zLAP reliability is assessed whenever zLAP estimates deviate from an acceptable range;

FIG. 4 summarizes a second illustrative technique performed in accordance with the general method of FIG. 4, wherein zLAP reliability is assessed only if zLAP estimates persistently deviate from the acceptable range;

FIG. 5 summarizes an exemplary procedure for use with the techniques of FIGS. 3 and 4 for calculating zLAP based on conductance values derived from impedance detection pulses;

FIG. 6 is a simplified, partly cutaway view, illustrating the pacer/ICD of FIG. 1 along with a set of leads implanted in the heart of the patient;

FIG. 7 is a functional block diagram of the pacer/ICD of FIG. 6, illustrating basic circuit elements that provide cardioversion, defibrillation and/or pacing stimulation in the heart and particularly illustrating components for corroborating zLAP estimates; and

FIG. 8 is a functional block diagram illustrating components of a device programmer of FIG. 7, and in particular illustrating a programmer-based zLAP corroboration components.

### DESCRIPTION OF THE PREFERRED EMBODIMENTS

The following description includes the best mode presently contemplated for practicing the invention. The description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

### Overview of Implantable Medical System

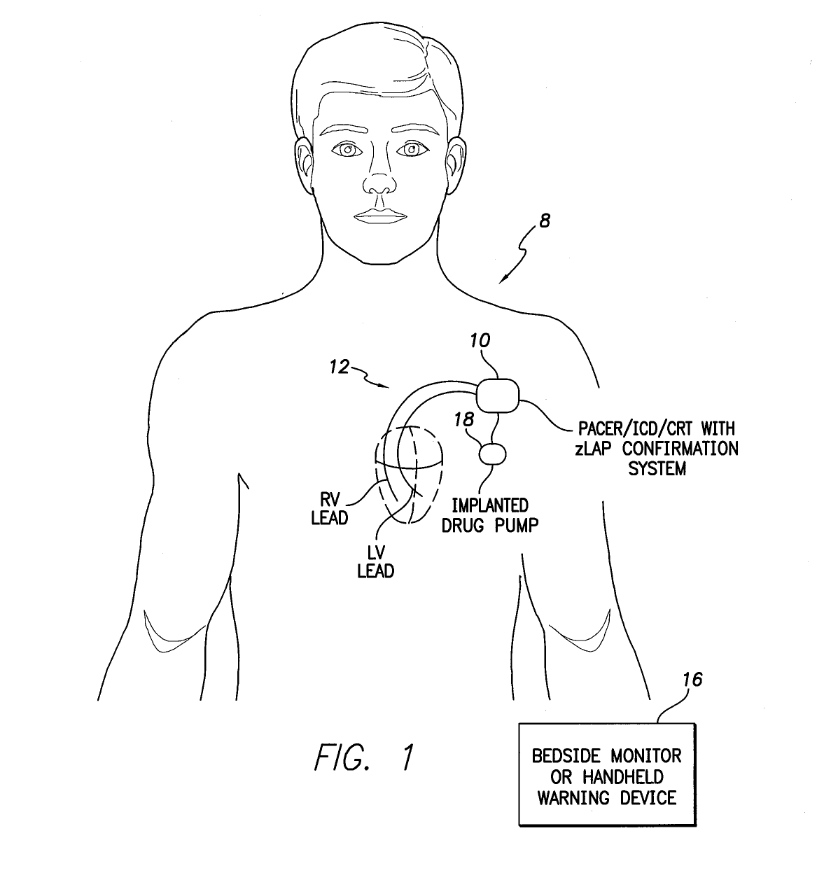

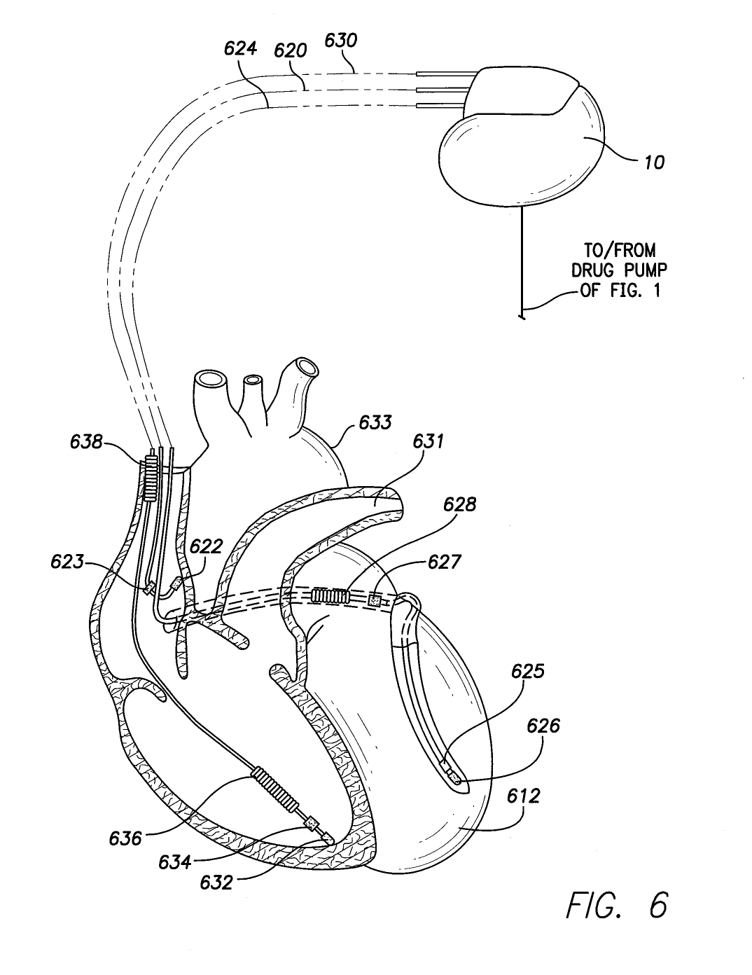

FIG. 1 provides a stylized representation of an exemplary implantable pacing medical system 8 capable of estimating LAP based on the impedance or related signals (i.e. capable of determining zLAP values) and further capable of confirming or corroborating its zLAP estimates based on various cardioelectric and cardiomechanical parameters. To this end, implantable medical system 8 includes a pacer/ICD/CRT device 10 or other cardiac stimulation device equipped to deliver impedance detection pulses using electrodes mounted to a set of sensing/pacing leads 12 and further equipped to determine zLAP values from signals sensed in response to the impedance detection pulses. For brevity herein, device 10 will be referred to as a pacer/ICD but it should be understood that other devices such as standalone CRT devices may instead be employed. Note also that in FIG. 1, only two leads are shown. A more complete set of leads is illustrated in FIG. 6, which is discussed below.

Within exemplary implementations described herein, zLAP is determined based on conductance, admittance or impedance parameters derived from electrical signals detected in response to impedance detection pulses. Predetermined conversion factors are stored within the pacer/ICD for use in converting the various detected parameters into zLAP values or other appropriate cardiac pressure values. As will be explained, the device can additionally detect a variety of cardioelectric and cardiomechanical parameters, which are used to confirm the zLAP estimates and to suspend/cancel the further estimates in circumstances where zLAP is deemed unreliable.

The pacer/ICD can also be equipped to track changes in zLAP so as to detect and track HF and/or cardiogenic PE. In response to HF, CRT therapy may be initiated and controlled by the implanted device. Techniques for performing CRT are discussed in the patents to Mathis et al., Kramer et al., and Stahmann et al., cited above. CRT parameters may be adaptively adjusted to improve the effectiveness of CRT using techniques set forth in the Panescu et al. patent application, “Closed-Loop Adaptive Adjustment of Pacing Therapy based on Cardiogenic Impedance Signals Detected by an Implantable Medical Device,” cited above. Additionally or alternatively, the pacer/ICD can issue warning signals, if warranted. For example, if zLAP exceeds a threshold indicative of HF or is rapidly increasing toward the threshold, warning signals may be generated to warn the patient, either using an internal warning device (which can be part of the pacer/ICD) or using an external bedside monitor/handheld warning device 16. The internal warning device may be a vibrating device or a “tickle” voltage device that, in either case, provides perceptible stimulation to the patient to alert the patient so that the patient may consult a physician. In one example, once the warning is felt, the patient positions an external warning device above his or her chest. The handheld device, which might be a personal advisory module (PAM), receives short-range telemetry signals from the implanted device and provides audible or visual verification of the warning signal. The handheld warning device thereby provides confirmation of the warning to the patient, who might otherwise be uncertain as to the reason for the internally generated warning signal. For further information regarding this warning/notification technique, see U.S. patent application Ser. No. 11/043,612, of Kil et al., filed Jan. 25, 2005, now U.S. Pat. No. 7,502,644.

If a bedside monitor is provided, the bedside monitor provides audible or visual alarm signals to alert the patient or caregivers, as well as providing textual or graphic displays. In addition, any diagnostic information pertaining to the deteriorating cardiac condition of the patient is transferred to the bedside monitor or is stored within the pacer/ICD for subsequent transmission to an external programmer or internet network site (not shown in FIG. 1) for review by a physician or other medical professional. The physician may then prescribe therapies to address the condition. The physician may also adjust the operation of the pacer/ICD to activate, deactivate or otherwise control any therapies that are automatically applied. The bedside monitor may be directly networked with a centralized computing system, such as the HouseCall™ remote monitoring system or the Merlin.Net system of St. Jude Medical, for immediately notifying the physician of any significant increase in zLAP. Networking techniques for use with implantable medical systems are set forth, for example, in U.S. Pat. No. 6,249,705 to Snell, entitled “Distributed Network System for Use with Implantable Medical Devices.”

In addition to CRT, other forms of therapy may also be controlled by the pacer/ICD in response to changes in zLAP. In this regard, if the implanted system is equipped with a drug pump 18, appropriate medications may be automatically administered upon detection of a significant increase in zLAP due to heart failure or cardiogenic PE. For example, medications may be delivered directly to the patient via the drug pump, if warranted. Alternatively, if a drug pump is not available, the patient may be provided with instructions—generated depending on the zLAP values—specifying the dosage of various heart failure medications to be taken. Exemplary heart failure medications include angiotensin-converting enzyme (ACE) inhibitors such as captopril, enalapril, lisinopril and quinapril, diuretics, digitalis, nitrates, and other compounds. Depending upon the particular medication, alternative compounds (e.g., intravenous or subcutaneous agents) may be required for use in connection with an implantable drug pump. Routine experimentation may be employed to identify medications for treatment of heart failure or other conditions that are safe and effective for use in connection with an implantable drug pump. Dosages may be titrated based upon the severity of heart failure as determined from zLAP or other parameters.

In addition to the zLAP corroboration procedures to be discussed in detail below, various techniques may be employed to confirm the detection of heart failure (or other medical conditions) made by the pacer/ICD before drug therapy is delivered. Exemplary heart failure detection/evaluation techniques are set forth in: U.S. Pat. No. 6,748,261, entitled “Implantable medical device for and Method of Monitoring Progression or Regression of Heart Disease by Monitoring Interchamber Conduction Delays”; U.S. Pat. No. 6,741,885, entitled “Implantable Cardiac Device for Managing the Progression of Heart Disease and Method”; U.S. Pat. No. 6,643,548, entitled “Implantable Medical Device for Monitoring Heart Sounds to Detect Progression and Regression of Heart Disease and Method Thereof”; U.S. Pat. No. 6,572,557, entitled “System and Method for Monitoring Progression of Cardiac Disease State using Physiologic Sensors”; and U.S. Pat. No. 6,480,733, entitled “Method for Monitoring Heart Failure”, each assigned to Pacesetter, Inc.

Hence, FIG. 1 provides an overview of an implantable medical system equipped to estimate LAP based on impedance or related signals, confirm those estimates, and deliver appropriate warning/notification signals and therapy in circumstances where reliable LAP estimates indicate that such actions are warranted. Embodiments may be implemented that do not necessarily perform all of these functions. For example, embodiments may be implemented that estimate LAP and assess the reliability of the estimates but do not automatically initiate or adjust HF therapy. Moreover, systems provided in accordance with the invention need not include all of the components shown in FIG. 1. In many cases, for example, the system will include only a pacer/ICD/CRT and its leads. Implantable warning devices and drug pumps are not necessarily implanted. Some implementations may employ an external monitor for displaying warning signals without any internal warning device. These are just a few exemplary embodiments. No attempt is made herein to describe all possible combinations of components that may be provided in accordance with the general principles of the invention. In addition, note that the particular locations of the implanted components shown in FIG. 1 are merely illustrative and may not necessarily correspond to actual implant locations. Although internal signal transmission lines provided are illustrated in FIG. 1 for interconnecting various implanted components, wireless signal transmission may alternatively be employed, where appropriate.

### Overview of LAP Confirmation/Corroboration

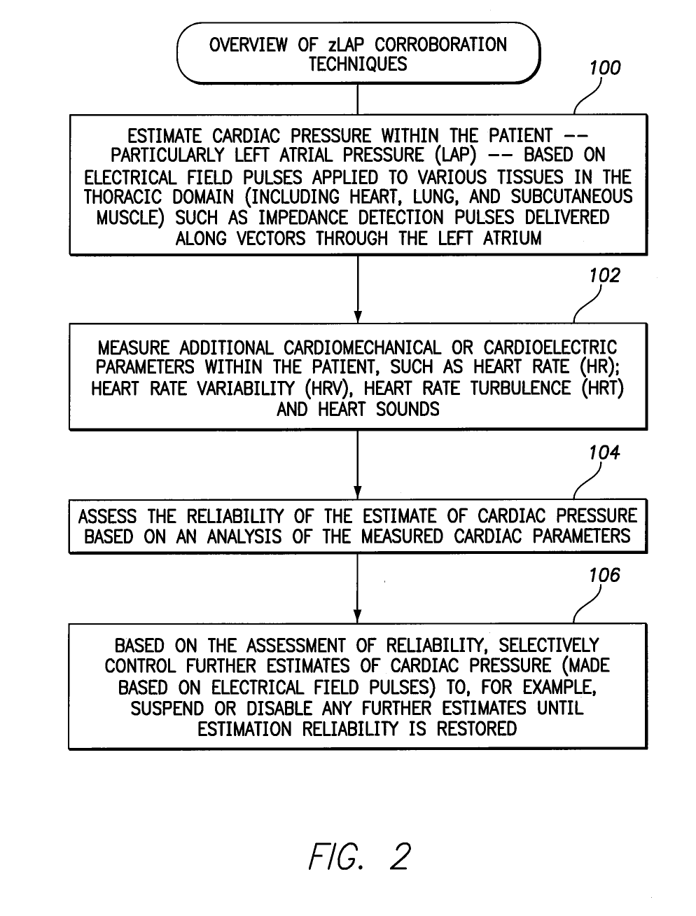

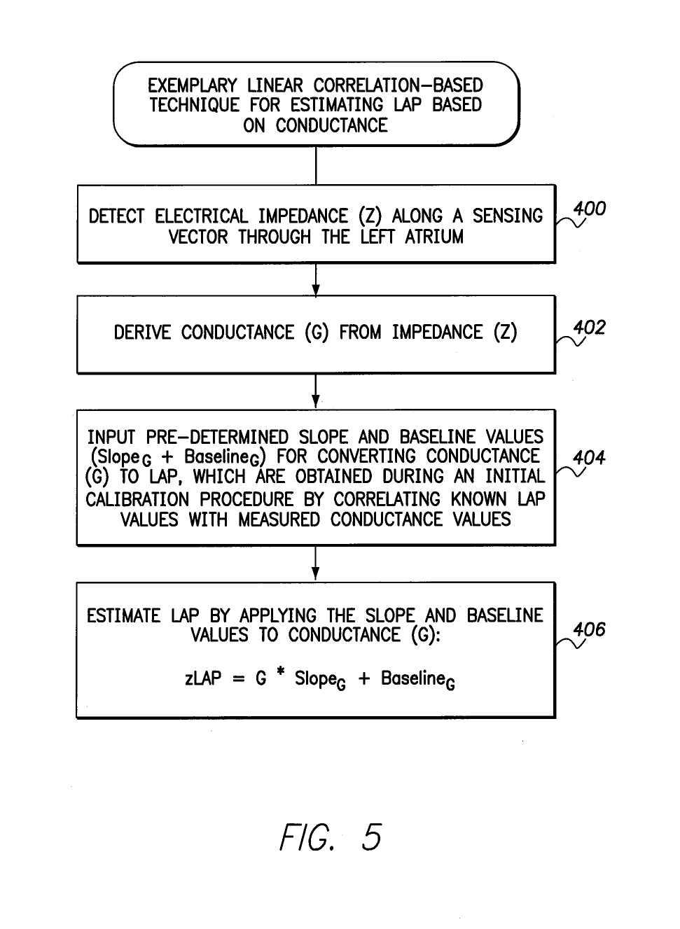

FIG. 2 provides an overview of the LAP estimate corroboration techniques that may be performed by the pacer/ICD of FIG. 1 or other suitably-equipped implantable device. At step 100, the device estimates cardiac pressure within the patient—particularly LAP—based on electrical field pulses applied to various tissues in the thoracic domain (that can include heart, lung, and subcutaneous muscle), such as impedance detection pulses delivered along vectors through the left atrium. That is, at step 100, the device determines zLAP. Exemplary zLAP determination techniques are described in the above-cited applications, such as U.S. patent application Ser. No. 11/559,235, now U.S. Pat. No. 7,794,404, incorporated by reference herein. An exemplary conductance-based zLAP technique is discussed below in connection with FIG. 5.

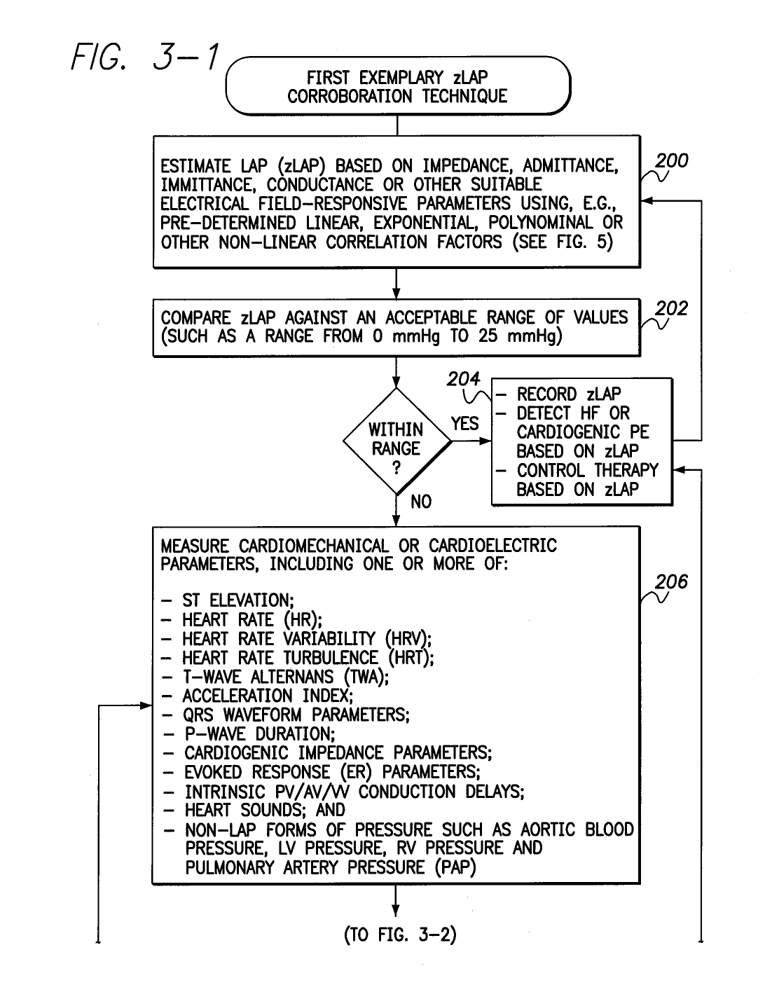

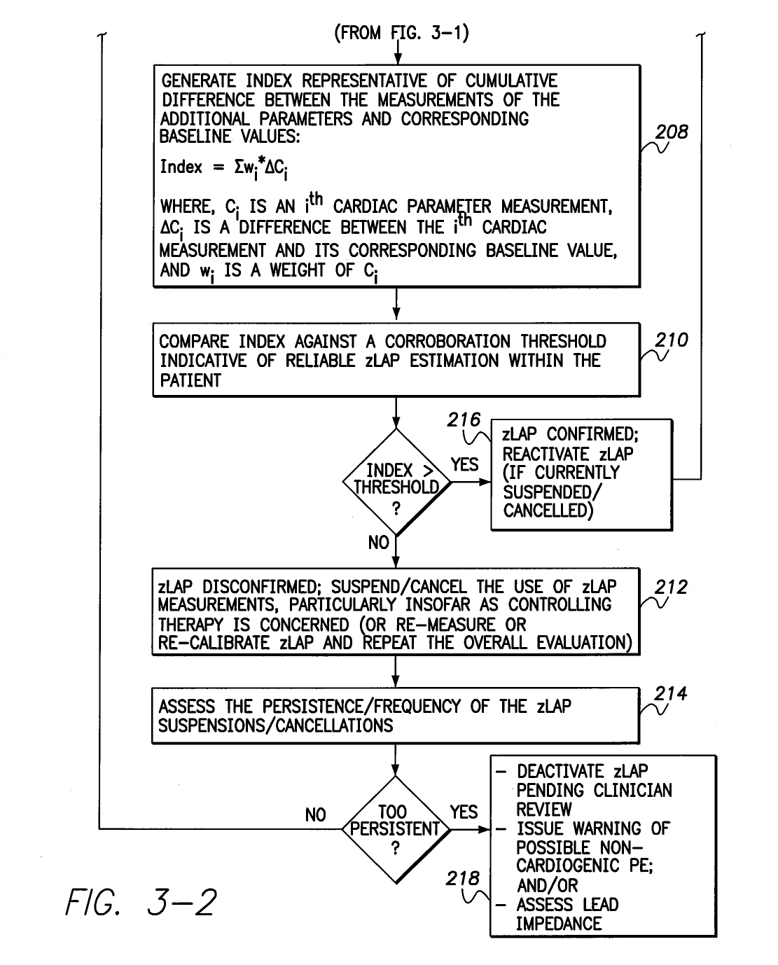

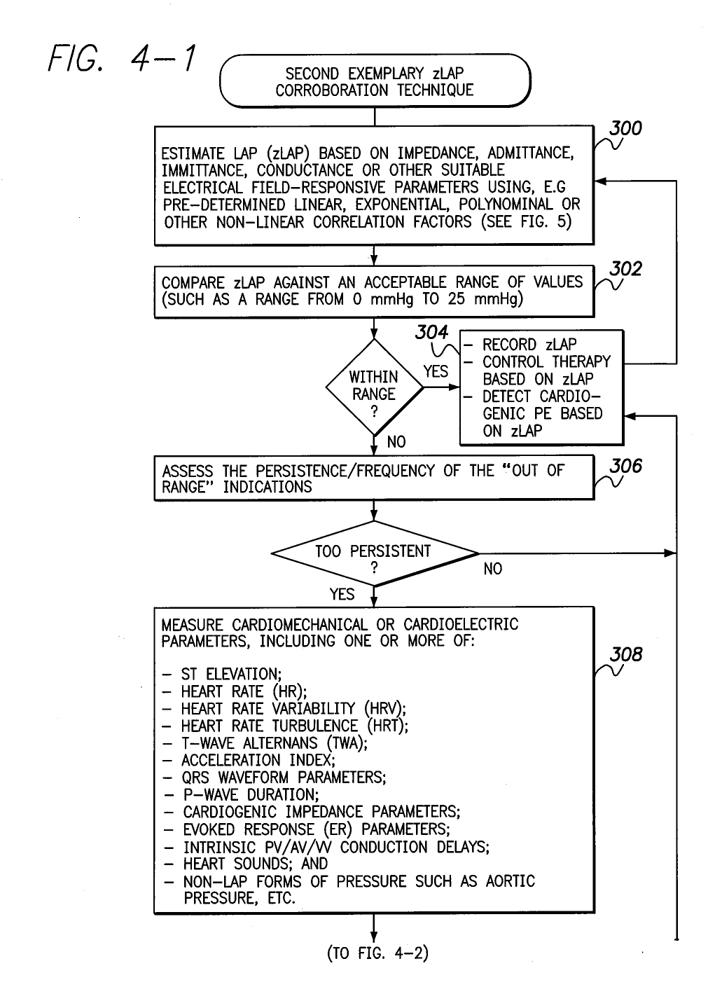

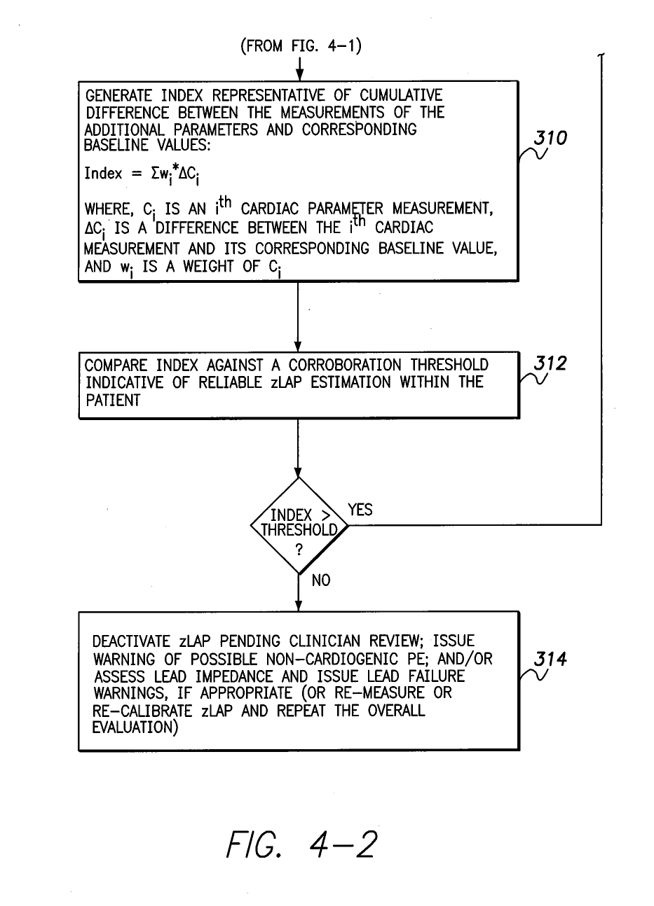

At step 102, the device measures additional cardiomechanical or cardioelectric parameters within the patient, such as heart rate (HR), heart rate variability (HRV), heart rate turbulence (HRT) and heart sounds. By “cardioelectric,” it is generally meant that the parameters are representative of—or are affected by—the electrical activity of the heart, such as parameters based on IEGM signals. Examples include ST segment elevation or QRS complex waveform parameters. By “cardiomechanical,” it is generally meant that the parameters are representative of—or are affected by—the mechanical activity of the heart, such as parameters measured by physiological sensors. Examples include heart sounds or arterial blood pressure. Note that some parameters—such as heart rate—can be derived either from cardioelectric signals (e.g. the IEGM) or from cardiomechanical signals (e.g. heart sounds.) Exemplary techniques for detecting these or other cardioelectric and cardiomechanical parameters are discussed below in connection with FIGS. 3 and 4.

At step 104, the device assesses the reliability of the estimate of cardiac pressure based on an analysis of the measured cardioelectric and cardiomechanical parameters. Exemplary techniques that exploit a cumulative index are discussed below in connection with FIGS. 3 and 4. Then, at step 106, based on the assessment of reliability, the device selectively controls further estimates of cardiac pressure (made based on the impedance pulses) to, for example, suspend, cancel or disable further estimates (or automatically re-measure or recalibrate cardiac pressure, if so equipped.) As will be explained, the device can selectively suspend or cancel further zLAP measurements until estimation reliability is restored or until clinician review. Additional actions may also be triggered, such as generating warnings indicative of possible noncardiogenic PE or triggering a lead failure test.

Exemplary zLAP Corroboration Techniques

Referring next to FIG. 3, a first illustrative zLAP corroboration technique will be described. In this example, the reliability of zLAP is assessed whenever the detected value for zLAP deviates from a predetermined range. Beginning at step 200, the pacer/ICD estimates LAP (i.e. the device determines zLAP) based on impedance, admittance, immittance, conductance or other suitable electrical field-responsive parameters, which can exploit pre-determined correlation factors including linear, exponential, polynomial or other non-linear correlation factors. (See the linear correlation-based example of FIG. 5, described below.) At step 202, the device compares zLAP against a preprogrammed or predetermined range of satisfactory, nominal, healthy or otherwise “acceptable” values, such as a range from 0 mmHg to 25 mmHg. In this regard, the device can be programmed to average some number of recent zLAP values for comparison against the upper and lower thresholds. These thresholds may be specified or adjusted by the clinician. Assuming that zLAP remains within the range, the reliability of the zLAP values is not questioned and, at step 204, the device performs various zLAP-responsive functions such as: recording zLAP values for diagnostic and trending purposes, detecting HF, cardiogenic PE or other cardiologic conditions such as MR based on trends in zLAP and/or controlling therapy based on trends in zLAP. See, e.g., the various HF detection techniques of the above-cited patent applications. In one example, a sharply increasing trend in zLAP toward the upper threshold (25 mmHG) might be deemed to be indicative of the onset of HF. A sharp increase in zLAP can be used to indicate PE.

However, if the zLAP estimate deviates from the acceptable range, then the device performs steps to assess the reliability of zLAP. More specifically, at step 206, the device measures various cardiomechanical or cardioelectric parameters including one or more of: ST elevation; heart rate (HR); heart rate variability (HRV); heart rate turbulence (HRT); T-wave alternans (TWA); an acceleration index; QRS waveform parameters; P-wave duration; non-LA cardiogenic impedance parameters; evoked response (ER) parameters; intrinsic PV/AV/VV conduction delays; heart sounds and non-LAP forms of blood pressure, such as aortic pressure. These are just some examples; other parameters may be used, as well, such as RV or LV pressures and pulmonary artery pressure (PAP).

ST elevation is measured within an IEGM signal detected by the device and represents the elevation of the portion of the signal between the end of a QRS-complex and the start of the subsequent T-wave. It can be measured relative to an IEGM baseline voltage. For ST elevation measurement techniques, see, e.g., U.S. patent application Ser. No. 12/016,166 of Boileau et al. filed Jan. 17, 2008, entitled “Systems and Methods for Distinguishing Cardiac Ischemia from Systemic Influences on IEGM Morphology using an Implantable Medical Device.” Heart rate (HR) is also easily measured from the IEGM, such as by detecting the rate of QRS-complexes (i.e. R-waves.)

HRV is a measure of the variation in heart rate over time. Exemplary techniques for measuring HRV are described in U.S. patent application Ser. No. 12/558,385, filed Sep. 11, 2009, of Bharmi et al., entitled “System and Method for use with an Implantable Medical Device for Detecting Stroke based on Physiological and Electrocardiac Indices.” Briefly, in one example described therein, HRV is assessed based on an analysis of R-R intervals, including various frequency components thereof.

HRT is generally regarded as a physiological response of the sinus node of the heart to premature ventricular contractions (PVCs.) Exemplary techniques for measuring HRT are also described in Bharmi et al., which can be based on various turbulence measurements obtained from arterial blood pressure signals in response to PVCs. PVCs may be detected within the IEGM. The arterial pressure may be detected using a suitable arterial blood pressure sensor.

T-wave alternans (TWA) pertain to a periodic beat-to-beat variation in the amplitude or shape of the T-wave in the IEGM (typically, high/low amplitudes occurring at odd/even beats.) TWA, which can be detected within the IEGM, is discussed, e.g., in U.S. Pat. No. 7,245,968 to Farazi et al., entitled “Implantable Cardiac Device Providing Rapid Pacing T wave Alternan Pattern Detection and Method.” See, also, U.S. Pat. Nos. 7,756,571; 7,738,956; 7,697,978; 7,620,448; and 7,599,733 and U.S. Published Patent Application No. 2009/0318822, all assigned to Pacesetter Inc.

Insofar as the acceleration index is concerned, this pertains to the acceleration of the heart wall and can be measured using a cardiac wall motion sensor. See, for example, U.S. Pat. No. 6,009,349 of Mouchawar et al., “System and Method for Deriving Hemodynamic Signals from a Cardiac Wall Motion Sensor.” In some examples, the acceleration index is defined as the peak acceleration of blood flow in the aorta.

A variety of QRS waveform parameters may be measured within the IEGM, such as the peak or width of the QRS, preferably using a ventricular IEGM that emphasizes the QRS. P-wave duration may also be measured within an IEGM, preferably using an atrial IEGM that emphasizes the P-wave. Insofar as cardiogenic impedance parameters are concerned, this refers to impedance values affected by the beating of the heart.

The evoked response (ER), which is an electrical signal triggered in response to pacing pulses, may be measured within the IEGM. See, for example, U.S. Pat. No. 6,473,647 to Bradley, entitled “Implantable Cardiac Stimulation Device For and Method of Monitoring Progression or Regression of Heart Disease by Monitoring Evoked Response Features” and U.S. Pat. No. 6,711,439, also to Bradley et al., entitled “Evoked Response Variability as an Indicator of Autonomic Tone and Surrogate for Patient Condition.”

Intrinsic PV/AV/VV conduction delays are conduction timing delays occurring within the heart, such as the delay between a P-wave and a subsequent QRS-complex (PV delay), the delay between an A-pulse and a subsequent QRS-complex (AV delay), or the delay between a V-pulse delivered to one ventricles and a subsequent QRS-complex observed within the other ventricle (VV delay). These delays may be measured within one or more IEGMs, such as within atrial and ventricular IEGMs.

Heart sounds generally correspond to the closure of valves within the heart and may be detected using acoustic sensors or accelerometers. Briefly, a first (S1) heart sound is associated with closure of the mitral valve. A second (S2) heart sound is associated with closure of the aortic and pulmonic valves. See, for example, U.S. Pat. No. 7,139,609, of Min et al., entitled “System and Method for Monitoring Cardiac Function via Cardiac Sounds using an Implantable Cardiac Stimulation Device.”

Finally, insofar as “non-LAP forms of blood pressure”, this refers to arterial or cardiac pressure values other than LAP pressure, which can be detected using one or more pressure sensors or pressure sensing techniques.

As can be appreciated, the device need not measure or detect each of the parameters listed in step 206. Indeed, in some examples, only a selected one of these parameters might be used to corroborate zLAP. Several different parameters are preferred to improve the specificity and robustness of the corroboration. For each parameter that is measured, a baseline for that measurement is also determined by, or input into, the device. For example, an average value for a given parameter might be measured within the patient on a daily, weekly, or other frequent period. Also for each parameter, a weight is determined by, or input into, the device for indicating the weight to be given that particular parameter in confirming zLAP. Typically, these weights are determined in advance and programmed into the device and may be specified by, or adjusted by, the clinician. Otherwise routine experiments can be used to determine appropriate values for use as the weights based on clinical studies from populations of patients.

At step 208, the device then generates an index representative of the cumulative difference between the measurements of the additional parameters and their corresponding baseline values:

Index=Σwᵢ*ΔCᵢ

where Cᵢ is an i^{th} cardiac parameter measurement, ΔCᵢ is a difference between the i^{th} cardiac measurement and its corresponding baseline value, and wᵢ is a weight of C. For example, i=1 might correspond to ST elevation, i=2 might correspond to HR, and so on.

At step 210, the device compares the index against a corroboration threshold. The value of the threshold may depend upon the particular parameters to be used and the weights thereof. The corroboration threshold is preferably determined in advance and programmed into the device and, as with the weights, can be specified by, or adjusted by, the clinician. Otherwise routine experiments can be used to determine an appropriate value for the corroboration threshold based on clinical studies from a population of patients for use with particular combinations of parameters to be used in the index.

At step 212, if the index does not exceed the corroboration threshold—indicating that the zLAP measurement are unreliable—the device suspends/cancels the use of zLAP measurements, particularly insofar as controlling therapy is concerned. In this regard, the index numerically represents or quantifies a set of concordant or corroborative factors that tend to increase in circumstances where LAP values are abnormal. In this regard, cardiogenic abnormalities—such as the presence of HF, cardiogenic PE, MR, ischemia, etc.—that cause LAP to become abnormal will likely cause the various cardioelectric and cardiomechanical parameters measured at step 206 to also become abnormal. Accordingly, the greater the index, the more likely it is that the abnormal zLAP value is truly representative of an abnormal LAP within the patient. Conversely, the lower the index, the more likely it is that the abnormal zLAP value is due to a confounding factor such as noncardiogenic PE and hence not properly representative of the true LAP of the patient. As one example, a change in ST segment elevation might be indicative of myocardial ischemia, which could be associated with an increase in actual LAP. Hence, a significant deviation in ST segment elevation from its baseline value tends to corroborate an abnormally high zLAP estimate. The lack of any significant deviation in ST segment elevation tends to disconfirm the abnormally high zLAP estimate. ST segment elevation is just one example of a corroborative factor. By employing an index that takes into account numerous corroborative factors, the reliability of the zLAP estimate can be properly assessed. In the alternative, at step 212, the device can re-measure zLAP or re-calibrate zLAP and then repeat the overall evaluation, if it is so equipped.

At step 214, the device assesses the persistence and/or frequency of the zLAP suspensions/cancellations. That is, if the device had previously suspended zLAP (due to the index remaining below its corroboration threshold) and then reactivated zLAP at a later time, the device determines whether the zLAP suspensions have been relatively rare or have instead been persistent. This may be determined, e.g., by counting the number of suspensions occurring over some period of time (one month, for example) and comparing it to a predetermined index persistence threshold. Assuming that the zLAP suspensions have not been persistent, processing returns to step 206 so the device can continue to monitor the cardioelectric and cardiomechanical parameters to determine whether zLAP should be reactivated. In this regard, steps 206-214 can be performed periodically in a loop, until either the cumulative index rises above its threshold (indicating that zLAP is again reliable) or until the suspension of zLAP is deemed to be persistent (indicating that zLAP estimates should be cancelled.)

If, following repeated iterations of steps 206-214, the suspension of zLAP is eventually deemed to be persistent, zLAP is deactivated at step 218 pending clinician review. Also, at step 218, warning signals indicative of a possible noncardiogenic PE are generated, either for the patient or clinician, or both, so that appropriate steps can be taken to determine if noncardiogenic PE is indeed occurring within the patient. Still further, given that the detection problems might be due to lead failure, a lead failure test may be triggered to detect a possible lead failure that might have contributed to, or caused, the zLAP problems.

Returning to step 210, if the index is found to be above the corroboration threshold (either initially or following some number of iterations of steps 206-214), the reliability of zLAP is confirmed at step 216 (and zLAP is reactivated if it had previously been suspended or cancelled) so that zLAP can be used at step 204 to control therapy, etc.

Thus, FIG. 3 describes the use of a corroboration index derived from cardioelectric and/or cardiomechanical parameters. The index is a weighted value indicating the number of cardiac measurements that are different from their respective baseline. If the index>threshold (where threshold may be varied based on total number of cardiac measurements) or most cardiac measurements show similar trend in change from baseline, then zLAP measurement is considered valid. However, if all or most cardiac measurements do not show a change from baseline (i.e. index<threshold), then that zLAP estimation is aborted and if this is found to happen repeatedly, then a notification to patient or physician will be generated suggesting potential noncardiogenic PE.

Referring now to FIG. 4, a second illustrative corroboration technique will be described. In this example, zLAP reliability is only assessed if zLAP persistently deviates from its predetermined range. Many of the steps of FIG. 4 are the same or similar to those of FIG. 3 and will not be described again in detail. Briefly, beginning at step 300, the pacer/ICD determines zLAP and, at step 302, compares zLAP against the predetermined range of acceptable LAP values. Assuming that zLAP remains within the range, the reliability of the zLAP values is not questioned and, at step 304, the device performs various zLAP-responsive functions. However, if the zLAP estimate deviates from the acceptable range, the device assess the persistence and/or frequency by which zLAP has deviated from that range. That is, if the device had previously detected zLAP values outside the range, the device determines whether those deviations have been relatively rare or instead have been persistent. This may be determined, e.g., by counting the number of zLAP “out of range” deviations occurring over some predetermined amount of time (one month, for example) and comparing it to a zLAP persistence threshold.

Assuming that the zLAP “out of range” deviations have not been persistent, processing continues at step 304 so the device can use the zLAP values. Conversely, if the “out of range” deviations of zLAP are deemed to be persistent, then the reliability of the zLAP estimate is assessed based on various cardiac parameters, at step 308. These parameters may be the same as those exploited in FIG. 4, such as ST elevation; HR; HRV; HRT, etc. Again, the device need not measure or detect each of the parameters. Rather, in some examples, only one or a few selected parameters might be used to assess the reliability of zLAP. A relatively large set of parameters is preferred to improve the robustness of the corroboration techniques. In any case, for each parameter measured, a baseline for that measurement is again used. Weights are also employed.

At step 310, the device then generates the corroboration index representative of cumulative difference between the measurements of the additional parameters and their corresponding baseline values. At step 312, the device compares the index against the corroboration threshold indicative of reliable zLAP estimation within the patient. At step 314, if the index does not exceed the threshold—indicating that the zLAP measurement might be unreliable—the device deactivates zLAP pending clinician review. Also, at step 314, warning signals indicative of a possible noncardiogenic PE can be generated and/or a lead failure test may be triggered. Conversely, if the index exceeds the threshold—indicating that the zLAP measurements are likely reliable—zLAP is thereby confirmed and processing returns to step 304. In the alternative, at step 314, the device can re-measure zLAP or re-calibrate zLAP and then repeat the overall evaluation, if it is so equipped.

Note that, in implementations where therapy is automatically delivered in response to HF/PE, the pacer/ICD might be equipped to employ at least one other detection technique to corroborate the detection of the medical condition before therapy is delivered. Techniques for detecting or tracking heart failure are set forth in the following patents and patent applications: U.S. Pat. No. 6,328,699 to Eigler et al. entitled “Permanently Implantable System and Method for Detecting, Diagnosing and Treating Congestive Heart Failure”; U.S. Pat. No. 6,970,742 to Mann et al., entitle “Method for Detecting, Diagnosing, and Treating Cardiovascular Disease”; U.S. Pat. No. 7,115,095 to Eigler et al. entitled “Systems and Methods for Detecting, Diagnosing and Treating Congestive Heart Failure”; U.S. patent application Ser. No. 11/100,008, filed Apr. 5, 2005, of Kil et al., entitled “System and Method for Detecting Heart Failure and Pulmonary Edema based on Ventricular End-Diastolic Pressure using an Implantable Medical Device,” now U.S. Pat. No. 7,437,192; U.S. patent application Ser. No. 11/014,276, filed Dec. 15, 2004, of Min et al., entitled “System and Method for Predicting Heart Failure based on Ventricular End-Diastolic Volume/Pressure using an Implantable Medical Device,” now U.S. Pat. No. 7,272,443; U.S. patent application Ser. No. 10/810,437, filed Mar. 26, 2004, of Bornzin et al., entitled “System and Method for Evaluating Heart Failure Based on Ventricular End-Diastolic Volume Using an Implantable Medical Device,” now U.S. Pat. No. 7,505,814; and U.S. patent application Ser. No. 10/346,809, filed Jan. 17, 2003, of Min et al., entitled “System and Method for Monitoring Cardiac Function via Cardiac Sounds Using an Implantable Cardiac Stimulation Device,” now U.S. Pat. No. 7,139,609. See also: U.S. Pat. No. 6,572,557, to Tchou et al.; U.S. Pat. No. 6,645,153, to Kroll et al., entitled “System and Method for Evaluating Risk of Mortality Due to Congestive Heart Failure Using Physiologic Sensors”, and U.S. Pat. No. 6,438,408 to Mulligan et al., entitled “Implantable Medical Device for Monitoring Congestive Heart Failure.”

Exemplary zLAP Determination Technique

Turning now to FIG. 5, for the sake of completeness, a technique for calculating zLAP in response to impedance detection pulses will be described in detail. This particular technique employs linear correlation using conductance values but, as noted, other techniques can be used as well to calculate zLAP. At step 400, the pacer/ICD detects electrical impedance (Z) along a sensing vector where impedance is affected by cardiac pressure, particularly LAP. For example, the cardiogenic impedance signal may be sensed between an LV tip electrode and an RA tip electrode such that the sensing vector passes through the LA. However, impedance signals sensed between other electrode pairs, such as the LV lead and the device may alternatively be utilized to indirectly estimate LAP under the presumption that, if these electrode pairs span the region containing the blood within pulmonary veins, then a resulting estimate of pulmonary venous pressure may be used as an estimate for LAP.

Impedance signals are obtained by transmitting a current between a pair of electrodes, and subsequently measuring the voltage between the same or another pair of electrodes. The impedance is calculated as the ratio of the measured voltage to the transmitted current. Preferably, a tri-phasic impedance pulse waveform is employed to sense the impedance signal. The tri-phasic waveform is a frequency-rich, low energy waveform that provides a net-zero charge and a net-zero voltage. An exemplary tri-phasic pulse waveform is described in detail in the related patent applications, cited above. Depending upon the particular sensing vector, it may be appropriate to filter the impedance signal to eliminate or reduce any noncardiogenic components, such as any components arising due to respiration or changes in body position or posture. Bandpass filtering is typically sufficient to filter out respiratory components. To the extent that it is relevant to the assessment of LAP, body position may be identified by two-dimensional accelerometer or three-dimensional accelerometer using suitable techniques. See, for example, techniques discussed in U.S. patent application Ser. No. 12,649,647, filed Dec. 30, 2009, entitled “Methods and Systems that Use Implanted Posture Sensor to Monitor Pulmonary Edema” (Attorney Docket No. A09P3013US1) and U.S. patent application Ser. No. 12/649,665, filed Dec. 30, 2009, entitled “Methods and Systems that Use Implanted Posture Sensor to Monitor Left Atrial Pressure and/or Inter-Thoracic Fluid Volume” (Attorney Docket No. A09P3013US2.)

Although the examples described herein are primarily directed to estimating LAP, other cardiac pressure values may alternatively be estimated, such as left ventricular pressure (LVP), by using impedance signals detected using appropriate sensing vectors (e.g., LV-tip electrode to RV-ring electrode or RV-Shock coil). Indeed, multiple impedance signals may be sensed using different sensing vectors passing through different chambers of the heart so as to permit the pacer/ICD to estimate cardiac pressure within different chambers of the heart, assuming appropriate conversion values have been determined and calibrated. To this end, the implanted system may be equipped, e.g., with multiple electrodes per lead or with multiple leads per chamber. Unipolar or bipolar sensing systems may be employed.

Depending upon the implementation, particular components of an initial raw impedance signal (Z₀) detected by the pacer/ICD are exploited, such as the high-frequency cardiogenic impedance signal (Z_{C}) representative of the beating of the heart of the patient, the low-frequency respiratory impedance signal (Z_{R}) representative of the respiration of the patient, or the ultra-low frequency circadian impedance signal representative of daily variations in the raw impedance signal (Z₀) or the low-frequency respiratory impedance signal (Z_{R}). Note that current state-of-the art pacer/ICDs do not typically include a detection circuit specifically for detecting circadian impedance variations. There is a cardiogenic detection circuit that extracts the cardiogenic component (Z_{C}) of the impedance signal (also referred to as cardiogenic impedance (CI)) from the raw impedance signal (Z₀) by substantially filtering out noncardiogenic components. There is a low frequency detection circuit that extracts the respiratory component (Z_{R}) of the impedance signal (also referred to as respiratory impedance (RI)) by substantially filtering out non-respiratory components. Circadian variations may be detected by storing the raw impedance values over a 24-hour period then processing the recorded raw values to extract circadian variations. In the predecessor applications cited above, the term “low-frequency raw impedance signal” was used to refer to the respiratory impedance signal (Z_{R}). Techniques for detecting or extracting the various components of the initial raw impedance signal are discussed in the cited applications.

At step 402, the pacer/ICD derives electrical conductance (G) from the detected electrical impedance signals. At step 404, the pacer/ICD inputs predetermined conversion factors from memory for converting conductance to LAP (or other cardiac pressure values). The conversion factors may be, e.g., predetermined slope and baseline values obtained during a calibration procedure employing linear regression. That is, during the calibration procedure, known LAP values are correlated with measured conductance values to determine slope and baseline values (or other appropriate “correlation factors.”) See, e.g., the various LAP patent application cited above, especially those of Gutfinger et al. and Panescu et al. Different conversion factors are typically required depending upon the particular parameters derived from the electrical impedance signal. That is, different slope and baseline values are used for a conductance-based estimation than for an admittance-based estimation. LAP values estimated using different techniques might be averaged together.

At step 406, the pacer/ICD then estimates LAP or other cardiac pressure values within the patient by applying the conversion factors retrieved from memory (at step 404) to the parameter(s) derived from the electrical impedance signal (at step 402). When using slope and baseline conversion factors, cardiac pressure may be generally estimated using:

Cardiac Pressure=Derived_Parameter*Slope+Baseline

where Derived_Parameter represents the parameter derived from the impedance signal, i.e. conductance, admittance, etc., and Slope and Baseline represent the conversion factors appropriate for use with the particular derived parameter. This formula assumes a linear relationship between cardiac pressure and the derived parameters, which is an appropriate presumption based on the particular parameters discussed herein, at least insofar as estimating LAP is concerned. Routine experimentation may be performed to determine whether a linear relationship is also suitable for use in estimating other particular cardiac pressure values, such as LVP, or is also suitable for use with other parameters that might be derived from the electrical impedance signal besides those specifically mentioned herein. Moreover, it should be understood that linear models need not necessarily be used, i.e. more sophisticated correlation models may instead by employed. Linear models are preferred in view of their simplicity.

Steps 400-406 may be repeated in a loop so as to update the estimated LAP. Depending upon the particular parameter used to estimate LAP, the estimates may be performed substantially in real-time so as to permit the pacer/ICD to continuously, or at least very frequently, calculate new LAP values. That is, in some implementations, a real-time LAP(t) function may be estimated so as to allow the pacer/ICD to track beat-to-beat changes in LAP. In particular, estimates of LAP based on conductance or admittance may potentially be performed substantially in real-time, assuming the pacer/ICD is appropriately configured. This allows the pacer/ICD to respond promptly to changes within the heart of the patient.

Note that the cardiac pressure value estimated using the techniques described herein is an effective intracardiac pressure (P_{eff}) not an absolute pressure. It represents the absolute intracardiac pressure less intrathoracic pressure:

P_{eff}=P_{intracardiac}−P_{intrathoracic}

That is, the effective pressure is a type of gauge pressure. Unless otherwise noted, all estimated cardiac pressure values discussed herein, particularly estimated LAP, are effective pressure values. In some examples described herein, the term “effective LAP” may be used as a reminder that effective pressures are used. In any case, effective pressure values are typically more useful from a clinical perspective than absolute pressure values.

See also, various LAP estimation techniques and other techniques described in: U.S. patent application Ser. No. 12/853,130, filed Aug. 9, 2010, entitled “Near Field-Based Systems and Methods for Assessing Impedance and Admittance for Use with an Implantable Medical Device” (Attorney Docket No. A10P1031); U.S. patent application Ser. No. 12/853,157, also filed Aug. 9, 2010, entitled “Systems and Methods for Estimating Left Atrial Pressure (LAP) in Patients with Acute Mitral Valve Regurgitation for Use by an Implantable Medical Device” (Attorney Docket No. A10P1032), as well as techniques described in U.S. patent application Ser. No. ______, filed ______, entitled “Systems and Methods for Exploiting Near-Field Impedance and Admittance for use with Implantable Medical Devices,” also of Gutfinger et al. (Attorney Docket No. A10P1031US01).

Although primarily described with respected to examples having a pacer/ICD, other implantable medical devices may be equipped to exploit the techniques described herein. For the sake of completeness, an exemplary pacer/ICD will now be described, which includes components for performing or controlling the various functions and steps already described.

### Exemplary Pacer/ICD

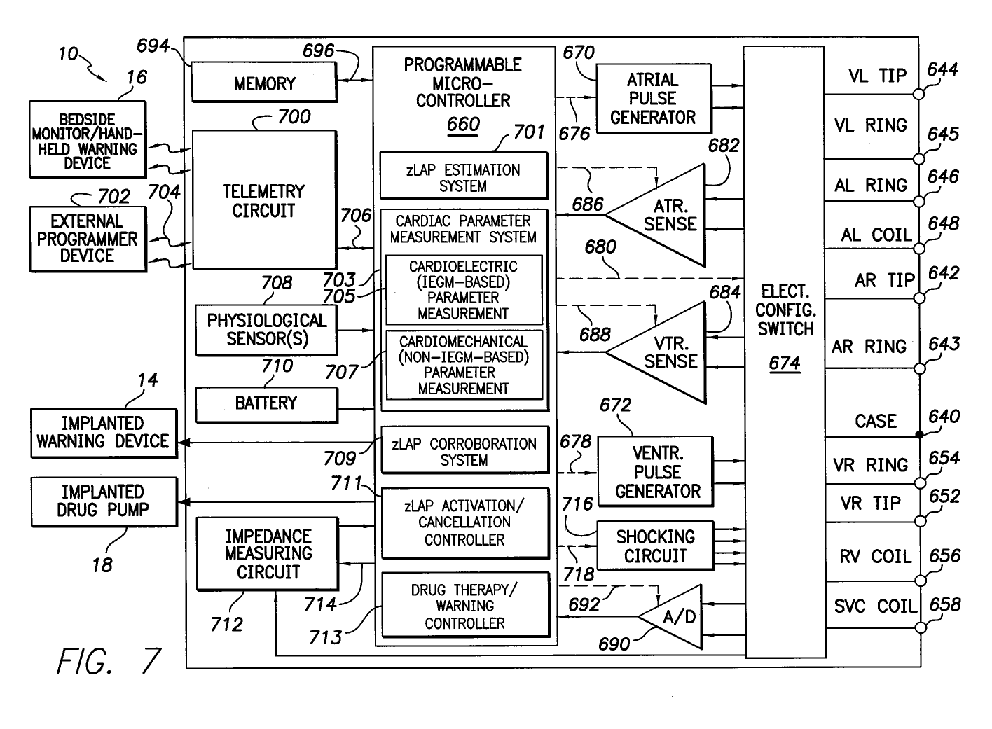

With reference to FIGS. 6 and 7, a description of an exemplary pacer/ICD will now be provided. FIG. 6 provides a simplified block diagram of the pacer/ICD, which is a dual-chamber stimulation device capable of treating both fast and slow arrhythmias with stimulation therapy, including cardioversion, defibrillation, and pacing stimulation, and also capable of estimating LAP or other forms of cardiac pressure using impedance signals. To provide other atrial chamber pacing stimulation and sensing, pacer/ICD 10 is shown in electrical communication with a heart 612 by way of a left atrial lead 620 having an atrial tip electrode 622 and an atrial ring electrode 623 implanted in the atrial appendage. Pacer/ICD 10 is also in electrical communication with the heart by way of a right ventricular lead 630 having, in this embodiment, a ventricular tip electrode 632, a right ventricular ring electrode 634, a right ventricular (RV) coil electrode 636, and a superior vena cava (SVC) coil electrode 638. Typically, the right ventricular lead 630 is transvenously inserted into the heart so as to place the RV coil electrode 636 in the right ventricular apex, and the SVC coil electrode 638 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a CS lead 624 designed for placement in the “CS region” via the CS is for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary CS lead 624 is designed to receive atrial and ventricular cardiac signals and to deliver left ventricular pacing therapy using at least a left ventricular tip electrode 626 and a LV ring electrode 625, left atrial pacing therapy using at least a left atrial ring electrode 627, and shocking therapy using at least a left atrial coil electrode 628. With this configuration, biventricular pacing can be performed. Although only three leads are shown in FIG. 6, it should also be understood that additional leads (with one or more pacing, sensing and/or shocking electrodes) might be used and/or additional electrodes might be provided on the leads already shown.

A simplified block diagram of internal components of pacer/ICD 10 is shown in FIG. 7. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 640 for pacer/ICD 10, shown schematically in FIG. 7, is often referred to as the “can”, “case” or “case electrode” and may be programmably selected to act as the return electrode for all “unipolar” modes. The housing 640 may further be used as a return electrode alone or in combination with one or more of the coil electrodes, 628, 636 and 638, for shocking purposes. The housing 640 further includes a connector (not shown) having a plurality of terminals, 642, 643, 644, 645, 646, 648, 652, 654, 656 and 658 (shown schematically and, for convenience, the names of the electrodes to which they are connected are shown next to the terminals). As such, to achieve right atrial sensing and pacing, the connector includes at least a right atrial tip terminal (A_{R} TIP) 642 adapted for connection to the atrial tip electrode 622 and a right atrial ring (A_{R} RING) electrode 643 adapted for connection to right atrial ring electrode 623. To achieve left chamber sensing, pacing and shocking, the connector includes at least a left ventricular tip terminal (V_{L} TIP) 644, a left ventricular ring terminal (V_{L} RING) 645, a left atrial ring terminal (A_{L} RING) 646, and a left atrial shocking terminal (A_{L} COIL) 648, which are adapted for connection to the left ventricular ring electrode 626, the left atrial ring electrode 627, and the left atrial coil electrode 628, respectively. To support right chamber sensing, pacing and shocking, the connector further includes a right ventricular tip terminal (V_{R} TIP) 652, a right ventricular ring terminal (V_{R} RING) 654, a right ventricular shocking terminal (V_{R} COIL) 656, and an SVC shocking terminal (SVC COIL) 658, which are adapted for connection to the right ventricular tip electrode 632, right ventricular ring electrode 634, the V_{R} coil electrode 636, and the SVC coil electrode 638, respectively.

At the core of pacer/ICD 10 is a programmable microcontroller 660, which controls the various modes of stimulation therapy. As is well known in the art, the microcontroller 660 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 660 includes the ability to process or monitor input signals (data) as controlled by a program code stored in a designated block of memory. The details of the design and operation of the microcontroller 660 are not critical to the invention. Rather, any suitable microcontroller 660 may be used that carries out the functions described herein. The use of microprocessor-based control circuits for performing timing and data analysis functions are well known in the art.

As shown in FIG. 7, an atrial pulse generator 670 and a ventricular pulse generator 672 generate pacing stimulation pulses for delivery by the right atrial lead 620, the right ventricular lead 630, and/or the CS lead 624 via an electrode configuration switch 674. It is understood that in order to provide stimulation therapy in each of the four chambers of the heart, the atrial and ventricular pulse generators, 670 and 672, may include dedicated, independent pulse generators, multiplexed pulse generators or shared pulse generators. The pulse generators, 670 and 672, are controlled by the microcontroller 660 via appropriate control signals, 676 and 678, respectively, to trigger or inhibit the stimulation pulses.

The microcontroller 660 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 674 includes a plurality of switches for connecting the desired electrodes to the appropriate I/O circuits, thereby providing complete electrode programmability. Accordingly, the switch 674, in response to a control signal 680 from the microcontroller 660, determines the polarity of the stimulation pulses (e.g., unipolar, bipolar, combipolar, etc.) by selectively closing the appropriate combination of switches (not shown) as is known in the art.

Atrial sensing circuits 682 and ventricular sensing circuits 684 may also be selectively coupled to the right atrial lead 620, CS lead 624, and the right ventricular lead 630, through the switch 674 for detecting the presence of cardiac activity in each of the four chambers of the heart. Accordingly, the atrial (ATR. SENSE) and ventricular (VTR. SENSE) sensing circuits, 682 and 684, may include dedicated sense amplifiers, multiplexed amplifiers or shared amplifiers. The switch 674 determines the “sensing polarity” of the cardiac signal by selectively closing the appropriate switches, as is also known in the art. In this way, the clinician may program the sensing polarity independent of the stimulation polarity. Each sensing circuit, 682 and 684, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits, 682 and 684, are connected to the microcontroller 660 which, in turn, are able to trigger or inhibit the atrial and ventricular pulse generators, 670 and 672, respectively, in a demand fashion in response to the absence or presence of cardiac activity in the appropriate chambers of the heart.

For arrhythmia detection, pacer/ICD 10 utilizes the atrial and ventricular sensing circuits, 682 and 684, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 660 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (ND) data acquisition system 690. The data acquisition system 690 is configured to acquire intracardiac electrogram signals, convert the raw analog data into a digital signal, and store the digital signals for later processing and/or telemetric transmission to an external device 702. The data acquisition system 690 is coupled to the right atrial lead 620, the CS lead 624, and the right ventricular lead 630 through the switch 674 to sample cardiac signals across any pair of desired electrodes. The microcontroller 660 is further coupled to a memory 694 by a suitable data/address bus 696, wherein the programmable operating parameters used by the microcontroller 660 are stored and modified, as required, in order to customize the operation of pacer/ICD 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 694 through a telemetry circuit 700 in telemetric communication with the external device 702, such as a programmer, transtelephonic transceiver or a diagnostic system analyzer. The telemetry circuit 700 is activated by the microcontroller by a control signal 706. The telemetry circuit 700 advantageously allows intracardiac electrograms and status information relating to the operation of pacer/ICD 10 (as contained in the microcontroller 660 or memory 694) to be sent to the external device 702 through an established communication link 704. Pacer/ICD 10 further includes an accelerometer or other physiologic sensor or sensors 708, sometimes referred to as a “rate-responsive” sensor because it is typically used to adjust pacing stimulation rate according to the exercise state of the patient.

However, physiological sensor(s) 708 can be equipped to sense any of the various cardiomechanical parameters discussed above, such HRT, heart sounds, etc. As can be appreciated, at least some these sensors may be mounted outside of the housing of the device and, in many cases, will be mounted to the leads of the device. Examples of physiological sensors that might be used with the device are described in: U.S. patent application Ser. No. 11/927,026, filed Oct. 29, 2007, of Nabutovsky et al., entitled “Systems and Methods for Exploiting Venous Blood Oxygen Saturation in combination with Hematocrit or Other Sensor Parameters for use with an Implantable Medical Device.”

Moreover, the physiological sensor 708 may further be used to detect changes in cardiac output, changes in the physiological condition of the heart, or diurnal changes in activity (e.g., detecting sleep and wake states) and to detect arousal from sleep. Accordingly, the microcontroller 660 responds by adjusting the various pacing parameters (such as rate, AV delay, V-V delay, etc.) at which the atrial and ventricular pulse generators, 670 and 672, generate stimulation pulses. While shown as being included within pacer/ICD 10, it is to be understood that the physiologic sensor 708 may also be external to pacer/ICD 10, yet still be implanted within or carried by the patient. A common type of rate responsive sensor is an activity sensor incorporating an accelerometer or a piezoelectric crystal, which is mounted within the housing 640 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 710, which provides operating power to all of the circuits shown in FIG. 7. The battery 710 may vary depending on the capabilities of pacer/ICD 10. If the system only provides low voltage therapy, a lithium iodine or lithium copper fluoride cell typically may be utilized. For pacer/ICD 10, which employs shocking therapy, the battery 710 should be capable of operating at low current drains for long periods, and then be capable of providing high-current pulses (for capacitor charging) when the patient requires a shock pulse. The battery 710 should also have a predictable discharge characteristic so that elective replacement time can be detected. Accordingly, appropriate batteries are employed.

As further shown in FIG. 7, pacer/ICD 10 is shown as having an impedance measuring circuit 712, which is enabled by the microcontroller 660 via a control signal 714. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 712 is advantageously coupled to the switch 774 so that any desired electrode may be used. The impedance measuring circuit 712 also detects the impedance signals discussed above to use in estimating LAP. That is, impedance measuring circuit 712 is an electrical impedance (Z) detector operative to detect an electrical impedance (Z) signal within the patient along at least one sensing vector wherein impedance is affected by cardiac pressure.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 660 further controls a shocking circuit 716 by way of a control signal 718. The shocking circuit 716 generates shocking pulses of low (up to 0.5 joules), moderate (0.5-10 joules) or high energy (11 to 40 joules or more), as controlled by the microcontroller 660. Such shocking pulses are applied to the heart of the patient through at least two shocking electrodes, and as shown in this embodiment, selected from the left atrial coil electrode 628, the RV coil electrode 636, and/or the SVC coil electrode 638. The housing 640 may act as an active electrode in combination with the RV electrode 636, or as part of a split electrical vector using the SVC coil electrode 638 or the left atrial coil electrode 628 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 7-40 joules), delivered asynchronously (since R-waves may be too disorganized), and pertaining exclusively to the treatment of fibrillation. Accordingly, the microcontroller 660 is capable of controlling the synchronous or asynchronous delivery of the shocking pulses.

Insofar as LAP estimation is concerned, the microcontroller includes a zLAP estimation system 701 operative to estimate LAP or other forms of cardiac pressure based on parameters derived from impedance signals using the techniques described above. That is estimation system is operative to: measure a predetermined parameter within patient tissues, the parameter being influenced by an electrical field applied to tissues of the patient including cardiac tissues, the parameter also being affected by cardiac pressure, and to then estimate cardiac pressure within the patient by applying predetermined conversion factors to the measured parameter. The microcontroller also includes a cardiac parameter estimation system 703 operative to measure one or more additional cardiac parameters within the patient, such as cardioelectric parameters detected using a cardioelectric (IEGM-based) parameter measurement system 705 and cardiomechanical parameters detected using a cardiomechanical (non-IEGM-based) parameter measurement system 707. The cardioelectric parameters may be obtained, for example, based on an analysis of IEGM signals received from ND converter 690. The cardiomechanical parameters may be obtained, for example, based on an analysis of physiological signals received from physiological sensors 708.

A zLAP corroboration system 707 is operative to assess the reliability of the estimate of cardiac pressure (i.e. zLAP) based on the measured cardiac parameters and to then corroborate/confirm or disconfirm the estimate. A zLAP activation/cancellation controller is operative to selectively control zLAP estimation system 701 based on the assessment of reliability using techniques described above or other suitable techniques.

Diagnostic data pertaining to zLAP can be stored in memory 694. Warning and/or notification signals are generated, when appropriate, by a warning controller 713 then relayed to the bedside monitor 16 or to external programmer 702 (or other external system) via telemetry system 700. Controller 713 is also equipped to control an implantable drug pump, if one is provided, to deliver appropriate medications. Terminals for connecting the implanted warning device and the implanted drug pump to the pacer/ICD are not separately shown.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

At least some of the techniques described herein can be performed by (or under the control of) a suitably-equipped external device. For the sake of completeness, an exemplary device programmer will now be described, which includes components for performing or controlling at least some of the functions and steps already described.

### Exemplary External Programmer

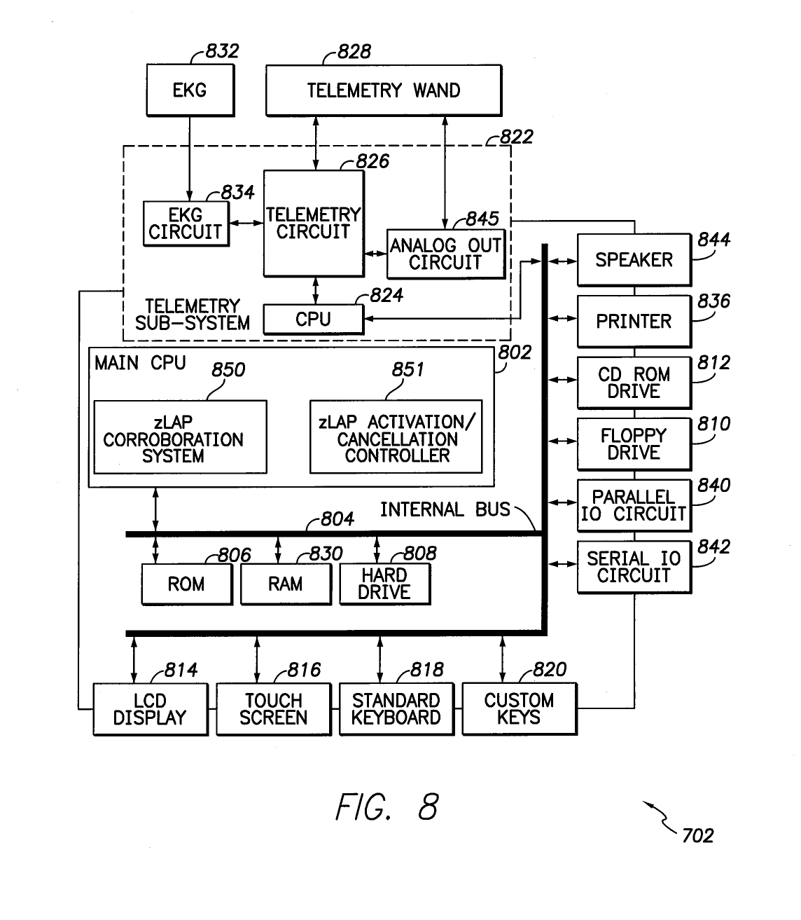

FIG. 8 illustrates pertinent components of an external programmer 702 for use in programming the pacer/ICD of FIG. 7 and for performing the above-described calibration techniques. For the sake of completeness, other device programming functions are also described herein. Generally, the programmer permits a physician or other user to program the operation of the implanted device and to retrieve and display information received from the implanted device such as IEGM data and device diagnostic data. Additionally, the external programmer can be optionally equipped to receive and display electrocardiogram (EKG) data from separate external EKG leads that may be attached to the patient. Depending upon the specific programming of the external programmer, programmer 702 may also be capable of processing and analyzing data received from the implanted device and from the EKG leads to, for example, render preliminary diagnosis as to medical conditions of the patient or to the operations of the implanted device.

Now, considering the components of programmer 702, operations of the programmer are controlled by a CPU 802, which may be a generally programmable microprocessor or microcontroller or may be a dedicated processing device such as an application specific integrated circuit (ASIC) or the like. Software instructions to be performed by the CPU are accessed via an internal bus 804 from a read only memory (ROM) 806 and random access memory 830. Additional software may be accessed from a hard drive 808, floppy drive 810, and CD ROM drive 812, or other suitable permanent mass storage device. Depending upon the specific implementation, a basic input output system (BIOS) is retrieved from the ROM by CPU at power up. Based upon instructions provided in the BIOS, the CPU “boots up” the overall system in accordance with well-established computer processing techniques.

Once operating, the CPU displays a menu of programming options to the user via an LCD display 814 or other suitable computer display device. To this end, the CPU may, for example, display a menu of specific programmable parameters of the implanted device to be programmed or may display a menu of types of diagnostic data to be retrieved and displayed. In response thereto, the physician enters various commands via either a touch screen 816 overlaid on the LCD display or through a standard keyboard 818 supplemented by additional custom keys 820, such as an emergency VVI (EVVI) key. The EVVI key sets the implanted device to a safe VVI mode with high pacing outputs. This ensures life sustaining pacing operation in nearly all situations but by no means is it desirable to leave the implantable device in the EVVI mode at all times.

Once all pacing leads are mounted and the pacing device is implanted, the various parameters are programmed. Typically, the physician initially controls the programmer 702 to retrieve data stored within any implanted devices and to also retrieve EKG data from EKG leads, if any, coupled to the patient. To this end, CPU 802 transmits appropriate signals to a telemetry subsystem 822, which provides components for directly interfacing with the implanted devices, and the EKG leads. Telemetry subsystem 822 includes its own separate CPU 824 for coordinating the operations of the telemetry subsystem. Main CPU 802 of programmer communicates with telemetry subsystem CPU 824 via internal bus 804. Telemetry subsystem additionally includes a telemetry circuit 826 connected to telemetry wand 828, which, in turn, receives and transmits signals electromagnetically from a telemetry unit of the implanted device. The telemetry wand is placed over the chest of the patient near the implanted device to permit reliable transmission of data between the telemetry wand and the implanted device. Herein, the telemetry subsystem is shown as also including an EKG circuit 834 for receiving surface EKG signals from a surface EKG system 832. In other implementations, the EKG circuit is not regarded as a portion of the telemetry subsystem but is regarded as a separate component.

Typically, at the beginning of the programming session, the external programming device controls the implanted devices via appropriate signals generated by the telemetry wand to output all previously recorded patient and device diagnostic information. Patient diagnostic information includes, for example, recorded IEGM data and statistical patient data such as the percentage of paced versus sensed heartbeats. Device diagnostic data includes, for example, information representative of the operation of the implanted device such as lead impedances, battery voltages, battery recommended replacement time (RRT) information and the like. Data retrieved from the pacer/ICD also includes the data stored within the recalibration database of the pacer/ICD (assuming the pacer/ICD is equipped to store that data.) Data retrieved from the implanted devices is stored by external programmer 702 either within a random access memory (RAM) 830, hard drive 808 or within a floppy diskette placed within floppy drive 810. Additionally, or in the alternative, data may be permanently or semi-permanently stored within a compact disk (CD) or other digital media disk, if the overall system is configured with a drive for recording data onto digital media disks, such as a write once read many (WORM) drive.

Once all patient and device diagnostic data previously stored within the implanted devices is transferred to programmer 702, the implanted devices may be further controlled to transmit additional data in real time as it is detected by the implanted devices, such as additional IEGM data, lead impedance data, and the like. Additionally, or in the alternative, telemetry subsystem 822 receives EKG signals from EKG leads 832 via an EKG processing circuit 834. As with data retrieved from the implanted device itself, signals received from the EKG leads are stored within one or more of the storage devices of the external programmer. Typically, EKG leads output analog electrical signals representative of the EKG. Accordingly, EKG circuit 834 includes analog to digital conversion circuitry for converting the signals to digital data appropriate for further processing within the programmer. Depending upon the implementation, the EKG circuit may be configured to convert the analog signals into event record data for ease of processing along with the event record data retrieved from the implanted device. Typically, signals received from the EKG leads are received and processed in real time.

Thus, the programmer receives data both from the implanted devices and from optional external EKG leads. Data retrieved from the implanted devices includes parameters representative of the current programming state of the implanted devices. Under the control of the physician, the external programmer displays the current programmable parameters and permits the physician to reprogram the parameters. To this end, the physician enters appropriate commands via any of the aforementioned input devices and, under control of CPU 802, the programming commands are converted to specific programmable parameters for transmission to the implanted devices via telemetry wand 828 to thereby reprogram the implanted devices. Prior to reprogramming specific parameters, the physician may control the external programmer to display any or all of the data retrieved from the implanted devices or from the EKG leads, including displays of EKGs, IEGMs, and statistical patient information. Any or all of the information displayed by programmer may also be printed using a printer 836.

Additionally, CPU 802 can include a zLAP corroboration system 850 operative to assess the reliability of cardiac pressure estimates made by an implanted device based on cardiac parameters measured by the device and sent to the external programmer. Based on the reliability assessment, the system confirms/corroborates the zLAP estimate or disconfirms the estimate. The CPU can also include a zLAP activation/cancellation controller 852 operative to selectively control the zLAP estimation system of the implanted device (see FIG. 7) based on the assessment of reliability, by transmitting suitable control signals to the implanted device.

Programmer/monitor 702 also includes a modem 838 to permit direct transmission of data to other programmers via the public switched telephone network (PSTN) or other interconnection line, such as a T1 line or fiber optic cable. Depending upon the implementation, the modem may be connected directly to internal bus 804 may be connected to the internal bus via either a parallel port 840 or a serial port 842. Other peripheral devices may be connected to the external programmer via parallel port 840 or a serial port 842 as well. Although one of each is shown, a plurality of input output (IO) ports might be provided. A speaker 844 is included for providing audible tones to the user, such as a warning beep in the event improper input is provided by the physician. Telemetry subsystem 822 additionally includes an analog output circuit 845 for controlling the transmission of analog output signals, such as IEGM signals output to an EKG machine or chart recorder.

With the programmer configured as shown, a physician or other user operating the external programmer is capable of retrieving, processing and displaying a wide range of information received from the implanted devices and to reprogram the implanted device if needed. The descriptions provided herein with respect to FIG. 8 are intended merely to provide an overview of the operation of programmer and are not intended to describe in detail every feature of the hardware and software of the programmer and is not intended to provide an exhaustive list of the functions performed by the programmer.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using ASICs or the like.

In general, while the invention has been described with reference to particular embodiments, modifications can be made thereto without departing from the spirit and scope of the invention. Note also that the term “including” as used herein is intended to be inclusive, i.e. “including but not limited to.”

What is claimed is:

1. A method for use with an implantable medical device for implant within a patient, the method comprising:
estimating cardiac pressure within the patient based on electrical field signals applied to tissues of the patient in the thoracic domain;
measuring one or more additional cardiac parameters within the patient;
assessing the reliability of the estimate of cardiac pressure based on the measured cardiac parameters; and
based on the assessment of reliability, selectively controlling further estimates of cardiac pressure.

2. The method of claim 1 further including determining whether the estimates of cardiac pressure are within a predetermined acceptable range, and wherein the steps of assessing the reliability of the estimate of cardiac pressure and selectively disabling further estimates of cardiac pressure are performed only if the estimates of cardiac pressure deviate from the predetermined acceptable range.

3. The method of claim 2 wherein the cardiac pressure value being estimated is left atrial pressure (LAP).

4. The method of claim 2 further including assessing a degree of persistence by which the cardiac pressure estimates deviate from the acceptable range, and wherein the steps of assessing the reliability of the estimate of cardiac pressure and selectively disabling further estimates of cardiac pressure are performed only if the degree of persistence exceeds a persistence threshold.

5. The method of claim 1 wherein estimating cardiac pressure within the patient based on electrical field signals applied to tissues of the patient in the thoracic domain includes:
applying electrical field pulses along a sensing vector passing through at least some cardiac tissue of the patient;
measuring a resulting electrical field-responsive parameter including one or more of impedance (Z), admittance (Y) and conductance (G); and
estimating cardiac pressure based on the resulting electrical field responsive parameter.

6. The method of claim 5 wherein estimating cardiac pressure based on the resulting electrical field responsive parameter is performed using pre-determined correlation factors including one or more of linear correlation factors, exponential correlation factors, polynomial correlation factors and non-linear correlation factors.

7. The method of claim 6 wherein estimating cardiac pressure includes calculating:
Cardiac Pressure=Parameter*Slope+Baseline
wherein Slope and Baseline are conversion factors representative of a linear correlation between the measured electrical field-responsive parameter and the cardiac pressure.

8. The method of claim 1 wherein measuring additional cardiac parameters includes measuring one or more cardioelectric parameters and cardiomechanical parameters.

9. The method of claim 8 wherein measuring one or more cardiac parameters includes measuring one or more of: ST elevation; heart rate (HR); heart rate variability (HRV); heart rate turbulence (HRT); T-wave alternans (TWA); an acceleration index; QRS waveform parameters; P-wave duration; cardiogenic impedance; evoked response (ER); intrinsic PV/AV/VV conduction delays; heart sounds; pulmonary artery pressure (PAP), LV pressure, RV pressure and other non-LAP forms of blood pressure.

10. The method of claim 1 wherein assessing the reliability of the estimate of cardiac pressure based on the additional cardiac parameters includes determining whether the additional cardiac parameters corroborate the cardiac pressure estimates in circumstances when cardiac pressure estimates deviate from a predetermined acceptable range.

11. The method of claim 10 wherein determining whether the additional cardiac parameters corroborate the cardiac pressure estimates includes:
generating an index representative of a cumulative difference between measurements of the additional cardiac parameters and corresponding baseline values; and
comparing the index against a corroboration threshold indicative of reliable cardiac pressure estimation.

12. The method of claim 11 wherein generating the index includes calculating:
Index=Σwᵢ*ΔC₁
where, Cᵢ is an i^{th} cardiac parameter measurement, ΔCᵢ is a difference between the i^{th} cardiac measurement and its corresponding baseline value, and wᵢ is a weight of Cᵢ.

13. The method of claim 10 wherein, if the additional cardiac parameters corroborate the cardiac pressure estimates, generating an indicator confirming the cardiac pressure estimates.

14. The method of claim 13 wherein, if the cardiac pressure estimates are confirmed, selectively delivering therapy to the patient based on the cardiac pressure estimates.

15. The method of claim 10 wherein, if the additional cardiac parameters do not corroborate the cardiac pressure estimates, re-measuring values and re-estimating cardiac pressure.

16. The method of claim 10 wherein, if the additional cardiac parameters do not corroborate the cardiac pressure estimates, re-calibrating cardiac pressure estimation procedures.

17. The method of claim 1 wherein all of the steps are performed by the implantable medical device.

18. The method of claim 1 wherein at least some of the steps are performed by an external device based on signals received from the implantable medical device.

19. A system for use with an implantable medical device for implant within a patient, the system comprising:
a cardiac pressure estimation system operative to estimate cardiac pressure within the patient based on electrical field signals applied to tissues of the patient, including tissues in the thoracic domain;
a cardiac parameter measurement system operative to measuring one or more additional cardiac parameters within the patient;
a reliability assessment system operative to assess the reliability of the estimate of cardiac pressure based on the measured cardiac parameters; and
a cardiac pressure estimation controller operative to selectively control the cardiac pressure estimation system based on the assessment of reliability.

20. A system for use with an implantable medical device for implant within a patient, the system comprising:
means for estimating cardiac pressure within the patient based on electrical field signals applied to tissues of the patient including tissues in the thoracic domain;
means for measuring one or more additional cardiac parameters within the patient;
means for assessing the reliability of the estimates of cardiac pressure based on the measured cardiac parameters; and
means for selectively controlling further estimates of cardiac pressure based on the assessment of reliability.
